(19) United States
(12) Patent Application Publication
(10) Pub. No.: US 2007/0073333 A1
(43) Pub. Date: Mar. 29, 2007

(54) Low profile filter assembly for distal embolic protection

(75) Inventor: James Coyle, Corbeg (IE)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/236,308

(22) Filed: Sep. 26, 2005

(51) Int. Cl. A61M 29/00

(52) U.S. Cl. 606/200

(57) ABSTRACT

A filter assembly configured to protect against atheroembolization in a blood vessel. The assembly includes an elongate hollow shaft, a wire slidingly engaged inside the shaft, and an elastic filter membrane. A distal region of the wire is predisposed to form a laterally expanded shape when extended from the shaft distal end. The elastic filter membrane slidably clings around the shaft distal end and is connected to a wire distal end. The membrane is stretched across the blood vessel by the laterally expanded shape when the wire distal region is extended from the shaft distal end.

### FIELD OF THE INVENTION

The present invention relates generally to intraluminal devices, and more particularly, to catheter or guidewire assemblies that include filters for distal embolic protection during an interventional procedure.

### BACKGROUND OF THE INVENTION

Stenotic lesions form on the lumen walls of a blood vessel to create narrowings that restrict blood flow there through, and may comprise a hard, calcified substance and/or a softer thrombus material. Interventional catheterization procedures such as balloon angioplasty, stent deployment, atherectomy, and thrombectomy are well known and have proven effective in the treatment of such stenotic lesions. Such procedures require the insertion of a therapy catheter through a patient's vasculature, and efforts are continually being focused toward improving their efficiency and efficacy.

Recently, devices have been developed that address concerns relating to atheroembolization, which is the obstruction of blood vessels by stenotic debris that may be released during interventional catheterization therapies such as those previously mentioned. Distal protection devices (DPDs) represent one class of intravascular devices that can be used to prevent atheroembolization. One type of DPD is an occluder that is mounted on a guidewire or catheter. During a medical procedure to treat a stenotic lesion, an occluder may be positioned distal to a stenotic lesion to temporarily stop the flow of blood and any stenotic debris that may have become entrained in the blood. The contaminated blood is aspirated from the treated area before the occluder device is collapsed to permit resumption of blood flow.

Another type of DPD is a vascular filter that is mounted on a guidewire or a catheter. During a stenosis treatment, a guidewire-mounted filter may be positioned distal to a stenotic lesion to capture any embolic debris. Then, the treatment catheter may be slid over the shaft of the filter guidewire to perform an intervention. When practical, it may be preferable to use a filter instead of an occluder to prevent atheroembolization since filters do not cause hemostasis. Conventional filters are typically formed of a mesh or other porous material through which blood may permeate. A catheter shaft that supports a filter may include an hydraulic control lumen. When fluid is forced through the lumen, an inflatable member expands the filter across the blood vessel. Another type of catheter system that supports a self-expanding filter may include a sliding sheath to collapse and deploy the filter.

Other intravascular DPD's may utilize wires or other mechanisms to expand a filter into apposition with the wall of the blood vessel lumen. These other mechanisms can have a larger collapsed profile than is desirable for crossing a vessel narrowing to be treated, especially when the DPD is used to make the preliminary advancement into or across a stenosis. If a large-profile DPD is the first device to be inserted through a lesion, atheroembolic debris may be dislodged there from and allowed to flow downstream before the DPD can be deployed distally of the lesion. Thus a DPD having a low collapsed profile is desirable to prevent the potential problem described above.

It is also beneficial to perform a balloon angioplasty or other interventional catheterization procedure rapidly, so it is desirable to provide a catheter or guidewire that includes a low-profile atheroembolization prevention filter that may be simply and quickly deployed and collapsed. The present invention provides these and other desirable features and characteristics that will become apparent from the subsequent detailed description and the appended claims taken in conjunction with the accompanying drawings.

### BRIEF SUMMARY OF THE INVENTION

In one exemplary embodiment, a filter assembly is provided that is configured to protect against atheroembolization in a blood vessel lumen. The filter assembly comprises an elongate hollow shaft, a wire, and an elastic filter membrane. The wire is slidingly engaged inside the hollow shaft. A distal region of the wire is predisposed to form a laterally expanded shape when extended from the shaft distal end. The elastic filter membrane is formed around the shaft distal end and connected to the wire distal end, and is configured to stretch across the blood vessel lumen when the wire distal end is extended from the shaft distal end and forms the laterally expanded shape.

In another exemplary embodiment, a method is provided for protecting against atheroembolization in a blood vessel when performing an interventional catheterization process. First, a filter assembly is positioned distal to a therapy site in the blood vessel. Then, a wire distal region is slid outside of the distal end of a hollow shaft to thereby allow the wire distal region to form a laterally expanded shape and stretch an elastic filter membrane across the blood vessel lumen at a position that is distal to the therapy site.

### BRIEF DESCRIPTION OF THE DRAWINGS

The following drawings are illustrative of a particular embodiment of the invention and therefore do not limit the scope of the invention. They are presented to assist in providing a proper understanding of the invention. The drawings are not to scale and are intended for use in conjunction with the explanations in the following detailed description. The present invention will hereinafter be described in conjunction with the appended drawings, wherein like reference numerals denote like elements, and:

FIG. 1 is a side view depicting a filter assembly including a wire, a hollow shaft, and a filter membrane in accordance with the invention;

FIG. 2 is a longitudinal cross-sectional view of the filter assembly taken along line 2-2 of FIG. 1;

FIG. 3 is a cross-sectional view of the filter assembly taken along line 3-3 of FIG. 1;

FIG. 4 is a longitudinal cross-sectional view of a filter assembly in accordance with the invention, the assembly being positioned at a distal region in a blood vessel with respect to a stenotic lesion;

FIG. 5 is a longitudinal cross-sectional view of the filter membrane partially collapsed and partially expanded across a blood vessel;

FIG. 6 is a longitudinal cross-sectional view of the filter membrane fully expanded across a blood vessel;

FIG. 7 is a cross-sectional view of the wire and the filter membrane taken along line 7-7 in FIG. 6;

### DETAILED DESCRIPTION OF THE INVENTION

The following detailed description is merely exemplary in nature and is not intended to limit the invention or the application and uses of the invention. Furthermore, there is no intention to be bound by any expressed or implied theory presented in the preceding technical field, background, brief summary, or the following detailed description. The terms “distal” and “proximal” are used in the following description with respect to a position or direction relative to the treating clinician. “Distal” or “distally” imply a position distant from or in a direction away from the clinician. “Proximal” and “proximally” imply a position near or in a direction toward the clinician.

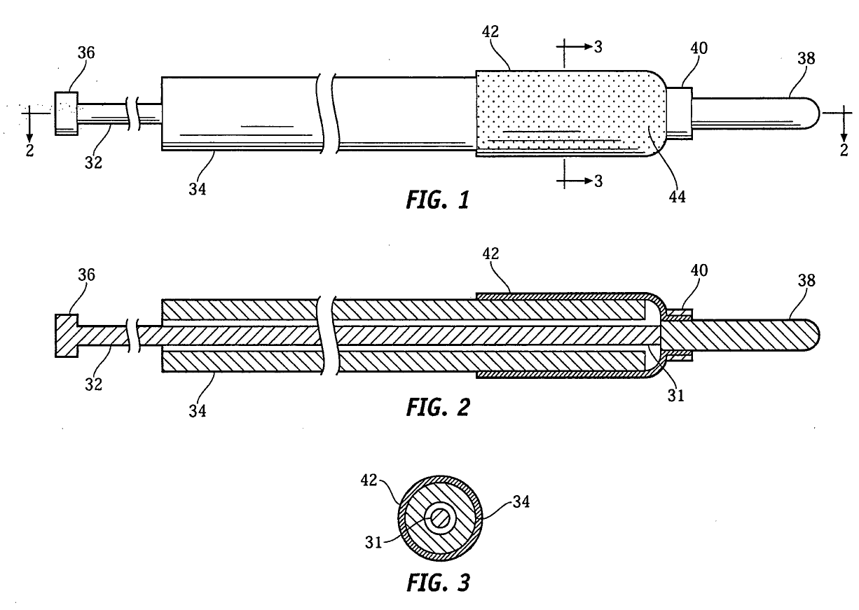

FIG. 1 is a side view depicting an exemplary filter assembly 100 that is adapted for use during an interventional catheterization procedure including but not limited to a balloon angioplasty, a stent deployment, an atherectomy, and a thrombectomy. FIG. 2 is a longitudinal cross-sectional view of the filter assembly taken along line 2-2 of FIG. 1, and FIG. 3 is a cross-sectional view taken along line 3-3 of FIG. 1. Filter assembly 100 includes wire 32, flexible tip 38 fixed adjacent a distal end of wire 32, and a hollow shaft 34 surrounding a portion of wire 32. Filter membrane 42 extends over distal region 31 of wire 32 and has a distal end attached adjacent a proximal end of tip 38. FIGS. 1 to 3 depict filter assembly 100 in an initial collapsed configuration when the filter membrane 42 is not deployed. In this configuration, hollow shaft 34 surrounds wire distal region 31, and filter membrane 42 is slidably clinging to an exterior surface of shaft 34. Handle 36 is optionally disposed at the proximal end of wire 32 to aid the operating clinician in grasping and manipulating wire 32. Handle 36 is no larger in diameter than shaft 34 in embodiments of the invention where shaft 34 is sized similarly to a medical guidewire, such that an interventional catheter can be slid there over.

Filter membrane 42 is an elastomer sleeve that is adapted to be stretched across the cross-sectional area of a blood vessel lumen. Various natural or synthetic elastic materials such as silicone or urethane may be utilized to form filter membrane 42. A plurality of pores formed through filter membrane 42 allows blood to flow through the membrane when it spans the blood vessel lumen.

The distal end of filter membrane 42 may be affixed to tip 38 by an adhesive joint, as well known by those of skill in the art of balloon catheters. Alternatively, or in addition, band 40 may be wrapped around the distal end of filter membrane 42 to secure it to tip 38. Band 40 may be a metal ring or an elastic band that constricts around the filter membrane distal end. Optionally, but not shown, the distal end of filter membrane 42 may be affixed directly to wire distal region 31 at a location near tip 38. The filter membrane proximal end is unattached to tip 38 or hollow shaft 34. However, in the initial collapsed configuration, the elastomer material is naturally contracted to form a low profile elastic sheath around hollow shaft 34.

Flexible tip 38 may be made of a flexible material and have a rounded atraumatic distal end to better lead filter assembly 100 through the curves and bends in a patient's vasculature. Techniques for assembling tip 38 and wire 32 are well known to those of skill in the art of medical guidewires. Tip 38 may comprise a soft polymer or a coil of fine wire. The portion of wire 32 that is disposed within tip 38 may be tapered to increase flexibility in the distal direction. The distal end of wire 32 and surrounding tip 38 and may be manually shapeable to form a bent tip (not shown) that can be steered from outside the patient's body by rotation of wire 32.

Wire distal region 31 is predisposed to take upon a laterally expanded shape, such as a spiraling coil, to which the distal region will revert when unconstrained by hollow shaft 34. Wire 32 is constructed of a material having the ability to recover to an original pre-formed shape after being temporarily straightened or constrained. Further, wire distal region 31 is sufficiently stiff to expand to its pre-formed shape substantially unimpeded by the surrounding filter membrane 42. In other words, wire distal region 31 can take on its laterally expanded shape, drawing or peeling filter membrane 42 off of hollow shaft 34 and expanding membrane 42 into apposition with the vessel wall. Exemplary wire materials include nitinol (TiNi), stainless steel, and high-modulus plastic, although other suitable materials may be used. In one embodiment, the wire 32 is a unitary filament with the desired expanded shape heat set directly into at least distal region 31. In an alternative embodiment, wire distal region 31 is separately manufactured and pre-formed with the desired laterally expanded shape. Then, wire distal region 31 is attached to the remaining wire portion by soldering, welding or other suitable joining means. For such an embodiment, wire distal region 31 and the remaining portion of wire 32 may be made from either the same or different materials.

Hollow shaft 34 is sufficiently flexible to navigate a patient's tortuous blood vessels while being sufficiently rigid to substantially straighten wire distal region 31 that the shaft surrounds, and to prevent surrounded wire distal region 3 from reverting to its pre-formed, laterally expanded shape. As with all of the filter assembly components, hollow shaft 34 is made of a biocompatible material. Shaft 34 may be made of thin-walled “hypotubing,” of stainless steel, nitinol, precipitation hardenable cobalt-based super alloy or other metals. Alternatively, shaft 34 may be made of high-modulus polymer such as polyimide or other thermoset resin. An exemplary hollow shaft 34 has an inner diameter ranging between about 0.008 and 0.010 inch, and has an outer diameter of approximately 0.014 inch. Such dimensions, along with a length of approximately 180 cm, can make this shaft useful in constructing a filter guidewire compatible with guidewire lumens of small diameter interventional catheters such as those used for percutaneous transluminal coronary angioplasty (PTCA). In such an embodiment, wire 32 has a diameter that is slightly less than 0.008 inch to allow the wire 32 to be slidably advanced and retracted through hollow shaft 34.

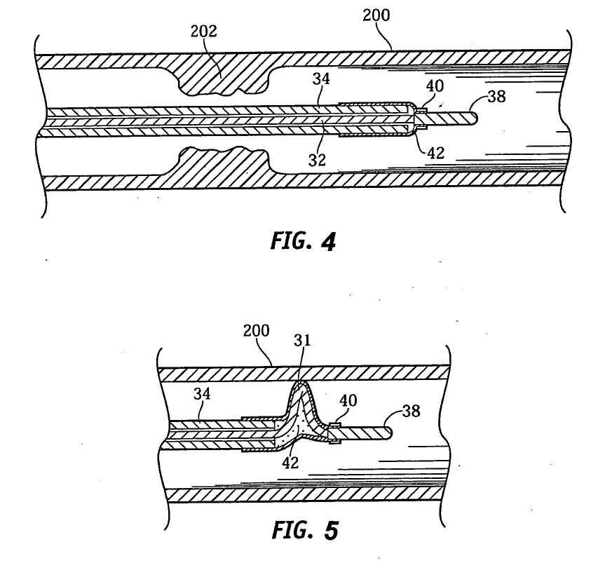

A method of using filter assembly 100 during an interventional catheterization procedure will be described next with particular detail to filter membrane 42 that provides distal embolic protection. FIG. 4 is a longitudinal cross-sectional side view of the filter assembly in blood vessel 200.

Filter membrane 42 remains in a self-contracted, non-deployed state while carried on filter assembly 100 to the desired location distal to lesion 202, as shown in FIG. 4. As previously mentioned, all but the distal end of filter membrane 42 is unattached, or not affixed to other elements of filter assembly 100. However, membrane 42 forms a low profile, slidable or peelable removable elastic sheath around hollow shaft 34.

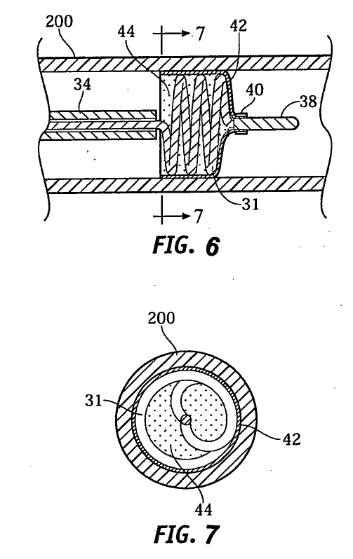

FIGS. 5 and 6 are longitudinal cross-sectional views of filter assembly 100 being expanded into a deployed configuration across the lumen of blood vessel 200 by separating the wire distal end from the hypotube distal end. One way to cause such separation is by only advancing wire 32, and not hollow shaft 34. More particularly, with the proximal end of shaft 34 outside the patient, a physician grasps hollow shaft 34 and holds it in place while pushing handle 36 toward shaft 34 to thereby advance wire 32. Another way to cause such separation at the distal end of the device is by only retracting or withdrawing hollow shaft 34, and not wire 32. More particularly, a physician grasps handle 36 and holds it in place before pulling the proximal end of shaft 34 toward handle 36.

According to the embodiment depicted in FIGS. 5 and 6, wire distal region 31 is heat set or otherwise predisposed to form a spiraling coil when uninhibited by hollow shaft 34. An exemplary wire distal region 31 is predisposed to form between one and three coils, although wire distal region 31 may also be predisposed to be otherwise shaped when expanded. Extending wire distal region 31 from the distal end of hollow shaft 34 allows region 31 to form its predisposed laterally expanded configuration, which in turn causes filter membrane 42 to expand in diameter. Since exemplary filter membrane 42 is formed from an elastic material, the coiled wire laterally expands or stretches filter membrane 42 until it spans the blood vessel lumen cross-sectional area and forms a temporary seal against the vessel lumen wall. The seal between membrane 42 and the vessel wall prevents blood with entrained embolic debris from passing around filter assembly 100.

When filter membrane 42 is deployed, the distal end of filter membrane 42 remains secured to tip 38 or to wire distal region 31 adjacent tip 38. However, since filter membrane 42 is not adhered to hollow shaft 34, its proximal end is open to allow embolic debris to enter filter membrane 42 and be retained therein.

FIG. 7 is a cross-sectional view of wire 32 and filter membrane 42, taken along line 7-7 in FIG. 6. Filter membrane 42 is secured by coiled wire distal region 31 in apposition with the walls of blood vessel 200. To allow for continued blood flow, pores 44 are formed through filter membrane 42. An exemplary pore diameter is about 100 microns, although the diameter may be modified as long as blood, but not embolic debris can permeate the filter.

With filter membrane 42 deployed across blood vessel 200 distal to lesion 202, an interventional catheterization procedure may be performed. For example, a dilatation or stent delivery catheter may be slid over shaft 34 to perform a treatment procedure on lesion 202. The filter membrane 42 would remain deployed during the treatment so that any embolic debris freed during the procedure would be captured in filter membrane 42.

After the interventional procedure, filter assembly 100 can be collapsed around wire 32 by bringing the wire distal end and the shaft distal end together This transformation from the deployed configuration to a collapsed configuration can be performed by reversing either of the earlier-described procedures that caused filter membrane 42 to deploy across blood vessel 200. During collapse of filter membrane 42, hollow shaft 34 returns wire distal region 31 to a substantially straight configuration as wire distal region 31 is retracted back into the shaft distal end, which in turn permits elastic filter membrane 42 to collapse around straightened wire distal region 31. Since wire distal region 31 is retracted into hollow shaft 34 proximal end first, the proximal end of filter membrane 42 will be the first membrane part to pull away from the wall of vessel 200 and to contract towards the distal end of shaft 34, thus closing the open proximal end of filter membrane 42, as shown in FIG. 5. During collapse of filter membrane 42, the filter proximal end may initially contract around the distal end of shaft 34 or around wire 32 adjacent thereto. Further retraction of wire distal region 31 into hollow shaft 34 can cause filter membrane 42, as it contracts, to bunch up around wire 32 distal to shaft 34 and/or to collapse around and/or slide over the distal end of shaft 34. Closing the proximal opening of deployed filter membrane 42 traps any previously captured embolic debris within the membrane for removal from the patient.

From the preceding description it is clear that the present invention provides an improved filter assembly configured for performing an interventional procedure within a patient's vasculature, and a method of providing embolic protection by distal filtration during such a procedure. Furthermore, the catheter assembly provides a push-pull, mechanically-operated filter assembly that includes a self-expanding coil extendable within an elastic filter membrane to enable fast and simple deployment of the filter assembly.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

What is claimed is:

1. A filter assembly deployable to protect against atheroembolization in a blood vessel, the filter assembly comprising:
an elongate hollow shaft terminating in proximal and distal ends;
an elongate wire slidingly engaged inside the hollow shaft and having proximal and distal ends extending there from, a distal region of the wire adjacent the wire distal end being predisposed to form a laterally expanded shape and having sufficient axial flexibility to have the laterally expanded shape substantially straightened when the wire distal region is inside the hollow shaft; and
an elastic filter membrane extending over the wire distal region and having a membrane distal end connected to the wire distal end, wherein the filter assembly is capable of assuming:
a collapsed configuration wherein the wire distal region is disposed inside the hollow shaft and the membrane slidingly clings to an exterior surface of the hollow shaft, and
a deployed configuration wherein the wire distal region extends from the shaft distal end to form the laterally expanded shape, which stretches the membrane across the blood vessel.

2. The filter assembly according to claim 1, wherein the elastic filter membrane is unattached to the hollow shaft and is configured to disassociate from the shaft when stretched across the blood vessel.

3. The filter assembly according to claim 1, wherein the elastic filter membrane includes a plurality of pores that are configured to allow blood to flow therethrough when the membrane is stretched across the blood vessel.

4. The filter assembly according to claim 3, wherein each of the pores has a diameter of about 100 microns.

5. The filter assembly according to claim 1, further comprising:
a flexible tip attached to the wire distal end, and
wherein the membrane distal end is directly attached to the flexible tip and is thereby connected to the wire distal end.

6. The filter assembly according to claim 5, further comprising a band wrapped around the elastic filter membrane and securing the elastic filter membrane to the flexible tip.

7. The filter assembly according to claim 6, wherein the band is formed from an elastomer material.

8. The filter assembly according to claim 1, wherein the wire proximal end includes a handle that is configured to allow a user to push and pull the wire through the hollow shaft.

9. The filter assembly according to claim 1, wherein the laterally expanded shape comprises at least one coil.

10. The filter assembly according to claim 9, wherein the laterally expanded shape comprises a plurality of coils.

11. A method of protecting against atheroembolization in a blood vessel when performing an interventional catheterization process, the method comprising:
providing a filter assembly comprising:
an elongate hollow shaft terminating in proximal and distal ends,
an elongate wire slidingly engaged inside the hollow shaft and having proximal and distal ends extending there from, a wire distal region adjacent the wire distal end being predisposed to form a laterally expanded shape and having sufficient axial flexibility to have the laterally expanded shape substantially straightened when the wire distal region is inside the hollow shaft, and
an elastic filter membrane slidably clinging around the shaft distal end and connected to the wire distal end;
positioning the filter assembly across a therapy site in the blood vessel; and
extending the wire distal region from the shaft distal end, thereby allowing the wire distal region to form the laterally expanded shape that stretches the elastic filter membrane across the blood vessel distal to the therapy site.

12. The method according to claim 11, wherein the elastic filter membrane is unattached to the hollow shaft; and
extending the wire distal region from the shaft distal end causes the filter membrane to disassociate from the shaft when stretched across the blood vessel.

13. The method according to claim 11, wherein the elastic filter membrane includes a plurality of pores configured to allow blood to flow there through when the elastic filter membrane is stretched across the blood vessel.

14. The method according to claim 12, wherein the wire proximal end includes a handle and extending the wire distal region distally from the shaft distal end is performed by grasping the handle and forcing the shaft and the handle toward each other.

15. The method according to claim 11, wherein the filter assembly further comprises:
a flexible tip attached to the wire distal end,
wherein the elastic filter membrane is directly attached to the flexible tip and is thereby connected to the wire.

16. The method according to claim 15, wherein the filter assembly further comprises:
a band wrapped around the elastic filter membrane and securing the elastic filter membrane to the flexible tip.

17. The method according to claim 16, wherein the band is formed from an elastic material.

18. The method according to claim 11, wherein the wire distal region is predisposed to form at least one coil when extended from the shaft distal end.

19. The method according to claim 18, wherein the wire distal region is predisposed to form a plurality of coils when extended from the shaft distal end.
